SEQUENCE LISTING (1 sequence)

SEQ ID NO: 1 - DNA, 3306 bp, Mus musculus
atgttctgggtagtctgaggagagtatgaggcgagccgtggcccgggtacctctccgctcccggggagaaggcgcggccgtggctgccgccctctgagtcgcggcgccggcgaggccccggggcgcgcgcatggtgctggtgccgctcgggtgttgatcggcctgtcccctccctctcttcccctccccaccccccgcggtggtctcccctttcccaccccagcccctgcggagccatggctcggagtggctcctgcccgcacctgttgtgggacgtgaggaaaaggtcccttgggctggaggacccgtcccggctgaggagccgctacctgggaagaagagaatttatccaaagattaaaacttgaagcaactttaaatgtgcatgatggctgtgttaatacaatctgttggaatgacactggagaatatattttatctggctctgatgacactaaacttgtaattagtaatccatacagcagaaaggttttgacaaccatccgttcagggcatcgagcaaatatatttagtgcaaagtttttgccgtgcacagatgataagcagattgtgtcttgctctggagatggagtcatattttatactaacattgagcaagatgcagaaactaacagacagtgccaatttacgtgccattatggaactacttatgagattatgactgtaccaaacgacccttacacctttctgtcctgtggtgaagatggaactgttaggtggtttgacacacgcatcaaaaccagttgcacaaaagaagactgtaaagatgatattttaatcaactgtaggcgtgctgccacatctgtggctatttgtcccccagtaccatattaccttgctgtgggttgttctgacagctcagtacggatttatgatcggcgaatgctgggcacaagagctacagggaattatgcaggccgaggaactactggaatggttgctcgatttataccttctcatcttagtaacaaatcatgcagagtgacatcactgtgttacagtgaagatggtcaagagattcttgtcagttattcttcagactacatctatctttttgaccccaaagatgatactgcacgagaacttaaaactccttctgcagaggagaggcgagaagagttacgacagcctccagttaagcgcttgagacttcgtggtgattggtcagatactggtcccagagcacggccagaaagtgaacgagaacgagatggagagcaaagtcccaatgtgtcactgatgcagagaatgtctgatatgttatcaaggtggtttgaagaagcaagtgaagttgcacaaagcaacagaggaagaggaagacctcggcccagaggtggaacaaatcagccagatgtttcaactcttcctacggttccatcaagtcctaatttggaagtgtgtgaaactgcaatggatgtagacatgccagctgcacttcttcagccttctacatcctctacagatccagttcaggctcaggcagccacagccgccatagaaagccctcgttccagctcgttgctgtcttgcccagacagtgaaccgaggcagtctgttgaggcgtctggacaccatgcacatcatcagtcagataacagtaatgagaggctgagccccaaaccagggacaggtgaacccgttttaagtttgcactacagcacagaaggaacaactacaagcacaataaaactgaactttacagatgaatggagcagtacagcctcaagttccagaggaaatgggagccattgcaaatctgagggtcaggaagaatgcttggtccctccgagctctgtgcagccaccggaaggagacagtgaaacaagagctcctgaagaactatcagagaaaggaacacttccagaaaacctcactcaaaaccagatagatacagcacaacttgataacttcccagctgagccattggattctaactcaggagagaagaataacccaagtcaggacagcccttgtgggcttccagaagaaggcactttgtctgaaacagacagggagacttgtgagcaggccagcactgagagtgctaccaggcatgctagcaccaagcctgaactcccatcccagacagaagccattgagcaggccagcactgagagtgctaccaggcataccagtgccaatcctgaactcccatcccagacagaagccatagcacctttagctcatgaagacccatctgccagggactctgctctccaggacacagatgacagcgatgatgatccggtcttgatccctggtgcaagataccgaacaggacctggtgatagacgctccgctgttgcccgcattcaggagttcttcaggaggagaaaagaaaggaaagaaatggaagagctggatactttgaacattaggaggccactagtaaagatggtttataagggccaccgcaactcccggacaatgataaaagaagccaatttctggggtgctaactttgtaatgagcggttccgattgtggccatatcttcatctgggaccggcacactgctgagcatttgatgcttctggaagctgataatcatgtggtcaactgcctgcagccccatccgtttgacccaattctagcctcatctggcatagattatgacataaagatctggtcgccactagaagagtcaagaatttttaatcgaaaacttgctgatgaagttataactcggaatgaactcatgctggaagagactcggaacaccatcaccgtcccagcctctttcatgttgaggatgttggcgtcactgaatcatatccgagctgaccgtctggagggtgacagatcagaaggttcaggtcaggagaatgaaaatgaggatgaagaataaagaactccttggcaagcacttagatgttctgagatttgtatacgacatttattatattttttttctttacagaactttagtgcaatttaaggctatgggttttttttttttttttttttttttggagttcttccctattttggggataaccaaacattggtttggaatgagtgtgtgcatgagttgggagagtgtgtaaaacaaagtaagcaaaatgttttttgaaaccttttgccgtgtatggagtccaaaaaaaaaaaaaaaaaaaaaaaaaaagcaaagtgcaatacttcctgaccctccgctgtgggagcttggatcaatgctgaagtcattttcattgtagtgaaaatgttggttcaaataaatttctacacttgccatttgcatgtttgttgctttctaattaaagatgttggttgctttaagatatcctaaa

(19) United States
(12) Patent Application Publication
(10) Pub. No.: US 2015/0226750 A1
(43) Pub. Date: Aug. 13, 2015

(54) NRIP AS A BIOMARKER OF ABNORMAL FUNCTION OF MOTOR NEURONS

(71) Applicant: NATIONAL TAIWAN UNIVERSITY, Taipei (TW)

(72) Inventors: Show-Li Chen, Taipei (TW); Hsin-Hsiung Chen, Taipei (TW); Yuan-Chun Huang, Taipei (TW)

(73) Assignee: NATIONAL TAIWAN UNIVERSITY, Taipei (TW)

(21) Appl. No.: 14/178,245

(22) Filed: Feb. 11, 2014

(51) Int. Cl. G01N 33/68

(52) U.S. Cl. CPC G01N 33/6875; G01N 2333/4703; G01N 2800/56; G01N 2800/285

(57) ABSTRACT

The present invention provides a method for evaluating physiological state of motor neurons in a subject. The method comprises detecting an expression level of a nuclear receptor interaction protein (NRIP) in a biological sample from the subject; and comparing the expression level of the NRIP to a control value for NRIP; wherein detecting a decrease of the expression level of the NRIP in the biological sample from the subject as compared to the control value for NRIP indicates an abnormal function of the motor neurons of the subject

### FIELD OF THE INVENTION

The present invention is related to a method for evaluating a physiological state of motor neurons in a subject.

### BACKGROUND OF THE INVENTION

Motor neurons (MNs) (also named motoneurons) are located in the ventral horn of the spinal cord and in brainstem nuclei, and send their axons out through ventral roots or cranial nerves to innervate skeletal muscles. In vertebrate, all the MNs are cholinergic. Means they all release neuron transmitter acetylcholine (ACh). Thus, the enzyme ACh is commonly used as a marker for MNs.

The motor neuron diseases (MNDs) are a group of progressive neurological disorders that destroy motor neurons, the cells that control essential voluntary muscle activity such as speaking, walking, breathing, and swallowing. Normally, messages from nerve cells in the brain (called upper motor neurons) are transmitted to nerve cells in the brain stem and spinal cord (called lower motor neurons) and from them to particular muscles. When there are disruptions in the signals between the lowest motor neurons and the muscle, the muscles do not work properly and gradually weaken and may begin wasting away and develop uncontrollable twitching. When there are disruptions in the signals between the upper motor neurons and the lower motor neurons, the limb muscles develop stiffness, movements become slow and effortful, tendon reflexes such as knee and ankle jerks become overactive. Over time, the ability to control voluntary movement can be lost.

MNDs are classified according to which motor neurons are being affected, upper motor neurons, lower motor neurons, or both. In adults, the most common MND is amyotrophic lateral sclerosis (ALS), which affects both upper and lower motor neurons. Primary lateral sclerosis (PLS) is a disease of the upper motor neurons, while progressive muscular atrophy (PMA) affects only lower motor neurons in the spinal cord.

There are no specific tests to diagnose most MNDs. Symptoms may vary among individuals and may be similar in the early stages of the disease, making diagnosis more difficult. For the treatment of the MNDs, there is no cure or standard treatment for the disease. Symptomatic and supportive treatments are ways which can help people be more comfortable to maintaining the quality of their lives.

A nuclear receptor interaction protein (NRIP) is a transcription factor that only expresses in cell nuclei. The NRIP was previously reported to express in human skeletal muscle and central nerve system including brain and spinal cord from Northern blotting of normal human tissue array. It is also reported that lack of NRIP gene expression in clinical muscular dystrophy and NRIP knockout mice display weaker muscle strength, indicating that the NRIP plays a role in muscle function (Zhang Y et al, Differential expression profiling between the relative normal and dystrophic muscle tissues from the same LGMD patient. J Transl Med, 2006, 4: 53).

A previously unaddressed need in connection with the aforementioned deficiencies and inadequacies exists in the art, especially the roles of the NRIP played in MNDs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows that nuclear receptor interaction protein (NRIP) expresses and is co-localized with choline acetyltransferase (ChAT) in lumbar spinal cord. (A) and (B) show that NRIP^{+/+} mice tissue protein is harvested and performed western blot analysis. The results show that the NRIPs are expressed in all spinal cord segments while NRIP^{-/-} mice did not detect. (C) shows the representative image of NRIP co-localizing with ChAT. For immunofluorescence, the lumbar spinal cord frozen sections from 12 weeks old NRIP^{+/+} and NRIP^{-/-} mice are stained. Panels from a to h are NRIP^{+/+} mice, and panels from i to p are NRIP^{-/-} mice. The panels a, b, c, d, i, j, k, l are magnified in the panels e, f, g, h, m, n, o, p respectively. ChAT: labeled in red (panels a, e, i, m); NRIP: labeled in green (panels, b, f, j, n); nuclei are labeled by DAPI and shown in blue (panels c, g, k, o); the overlaid images of the ChAT and the NRIP are shown in yellow (panels d, h, l, p). The results show that the NRIP and the ChAT in NRIP^{+/+} mice are co-localized in ventral horn spinal cord (panels d, h). In NRIP^{-/-} mice, the staining of the NRIP is not visible (panels j, n) and the ChAT expression (panels i, m) is not intact. Scale bar, 100 μm.

FIG. 2 shows that the deficient motor neurons (MNs) in spinal cords of NRIP^{-/-} mice. (A) shows that the representative image of ChAT and neuronal nuclei (NeuN) expression in NRIP^{+/+} mice and NRIP^{-/-} mice. Panels from a to h are NRIP^{+/+} mice, and panels from i to p are NRIP^{-/-} mice. The panels a, b, c, d, i, j, k, l are magnified in the panels e, f, g, h, m, n, o, p respectively. ChAT: labeled in green (panels a, e, i, m); NeuN: labeled in red (panels, b, f, j, n); nuclei are labeled by DAPI and shown in blue (panels c, g, k, o); the overlaid images of the ChAT and the NeuN are shown in yellow (panels d, h, l, p). Except for the loss of MNs, the MNs of the NRIP^{-/-} mice (panels l, p) in ventral horn spinal cord show defective phenotype compared to the NRIP^{+/+} mice (panels d, h). Scale bar, 200 μm. (B) shows the quantitative analysis of α-MNs which are detected by the ChAT and the NeuN double positive cells from part A showing that α-MNs are significantly lost in NRIP^{-/-} mice by student's t test. **P<0.01.

FIG. 3 shows the loss of motor neurons in NRIP^{-/-} mice spinal cord. The lumbar spinal cord frozen section is from NRIP^{+/+} mice and NRIP^{-/-} mice (N=3, age between 12 weeks to 16 weeks). (A) shows the representative image of cresyl violet (Niss1)-stained lumbar spinal cord. NRIP^{+/+} mice (panel a, magnified in panel b); and NRIP^{-/-} mice (panel c, magnified in panel d). Scale bars, 200 μm. (B) shows that the quantitative analysis of MNs is counted from part A. Each animal is stained at least twelve sections with a distance of 120 μm. The results show that MNs (red arrow pointed) are lost in NRIP^{-/-} mice. **P<0.01 by student's t test. (C) shows the immunohistochemistry result of the ChAT in the lumbar spinal cord. NRIP^{+/+} mice (panel a, magnified in panel b). NRIP^{-/-} mice (panel c, magnified in panel d). The results further confirm that MNs are lost in NRIP^{-/-} mice. Scale bars, 100 μm.

FIG. 4 shows that the loss of MNs in NRIP^{-/-} mice is age-related but not developmental cause. (A) shows the representative image of the ChAT and the NeuN expression in the spinal cords frozen section of NRIP^{+/+} mice and NRIP^{-/-} mice at age 6 week. Panels from a to h are NRIP^{+/+} mice, and panels from i to p are NRIP KO mice. The panels a, b, c, d, i, j, k, l are magnified in the panels e, f, g, h, m, n, o, p respectively. ChAT: labeled in green (in panels a, e, i, m); NeuN: labeled in red (panels, b, f, j, n); nuclei are labeled by DAPI and shown in blue (panels c, g, k, o); the overlaid images of the ChAT and the NeuN are showed in yellow (panels d, h, l, p). The results show that no significant difference in the morphology of motor neurons exists between NRIP^{+/+} mice (panels d, h) and NRIP^{-/-} mice (panels l, p). Scale bar, 200 μm. (B) shows that the quantitative analysis of α-MNs from part A shows no significant difference in the number of MNs at early age (6 weeks) between NRIP^{+/+} mice and NRIP^{-/-} mice by student's t test.

FIG. 5 shows the astrogliosis in the spinal cords of NRIP^{-/-} mice. (A) shows the representative images of GFAP and MAP2 expression at age 12-16 weeks. Panels from a to h are NRIP^{+/+} mice, and panels from i to p are NRIP^{-/-} mice. The panels a, b, c, d, i, j, k, l are magnified in the panels e, f, g, h, m, n, o, p respectively. MAP2: labeled in green (panels a, e, i, m); GFAP: labeled in red (panels, b, f, j, n); nuclei are labeled by DAPI and shown in blue (panels c, g, k, o). The results show that the GFAP positive glia cells are infiltrated to the ventral horn of spinal cords in NRIP^{-/-} mice. Scale bar, 200 μm. (B) shows the quantitative analysis of the GFAP expression from part A. Each animal is selected at least four random sections, the total pixels of GFAP in NRIP^{+/+} mice and NRIP^{-/-} mice ventral horn spinal cord are measured by ImageJ software. The results show that GFAP positive pixels in NRIP^{-/-} mice are twice more than NRIP^{+/+} mice. **P<0.0, by student's t test.

FIG. 6 shows the microgliosis in the spinal cord of NRIP^{-/-} mice. (A) shows the representative images of expression of Iba1 and NeuN at age 12-16 weeks. Panels from a to h are NRIP^{-/-} mice, and panels from i to p are NRIP^{-/-} mice. The panels of a, b, c, d, i, j, k, l are magnified in the panels of e, f, g, h, m, n, o, p respectively. Iba1: labeled in green (panels a, e, i, m); NeuN: labeled in red (panels, b, f, j, n); nuclei are labeled by DAPI and shown in blue (in panels c, g, k, o). The results show that Iba1 positive microglia cells are infiltrated to the ventral horn of spinal cords in NRIP^{-/-} mice. Scale bar, 200 μm. (B) shows the quantitative analysis of Iba expression from part A. The results show that microglia cells are activated in NRIP^{-/-} mice.

FIG. 7 shows the mechanism of NRIP role in ChAT expression in cholinergic cell line NG108. NG108 cells are induced to differentiate by 1 mM dbcAMP with low (1%) serum condition. (A) shows that the cell morphology from undifferentiated status to four days after differentiation is shown in panels a to e. The magnified view is shown on the upper right corner of each panel. (B) shows the expression of the NRIP and the ChAT during the differentiation process. The cell lysates are harvested at indicated times and analyzed by western blot. The results show that both the NRIPs and the ChAT proteins increased in NG108 differentiation.

FIG. 8 shows that the ChAT is down-regulated upon silenced NRIP expression. NG108 cells are infected with adenovirus encoding shNRIP and simultaneously induced to differentiate by 1 mM dbcAMP in low (1%) serum condition. After 48 hrs, the cell lysates are harvested. Lane 2 shows the less of the ChAT expression upon knock-down of NRIP in cells.

### SUMMARY OF THE INVENTION

The present invention is directed to a method for evaluating a physiological state of motor neurons in a subject, comprising the following steps: detecting an expression level of a nuclear receptor interaction protein (NRIP) in a biological sample from the subject; and comparing the expression level of the NRIP to a control value for NRIP; wherein detecting a decrease of the expression level of the NRIP in the biological sample from the subject as compared to the control value for NRIP indicates an abnormal function of the motor neurons of the subject.

### DETAILED DESCRIPTION OF THE INVENTION

The present invention shows that nuclear receptor interaction protein (NRIP) can mediate a choline acetyltransferase (ChAT) expression level and can further affect the generation of acetylcholine (ACh). In addition, the present invention also demonstrates that the NRIP affects the differentiation of the motor neurons (MNs) by mediating the ChAT expression level. Based on above situation, the degeneration of the motor neurons is accompanied by the generation of gliosis.

According to above results, the present invention can serve as a diagnostic marker and a therapeutic target for diseases relating to motor neurons which are caused by NRIP deficiency. The present invention can be applied as follows: to make a pharmaceutical composition of NRIP used to treat motor neuron diseases, to use other agent repairing the abnormal function of NRIP to cure the motor neuron diseases caused by loss of NRIP, and to develop NRIP as a biomarker to identify the occurring risk or progress of motor neuron diseases.

As used herein in the specification, “a” or “an” can mean one or more. As used herein in the claim(s), when used in conjunction with the word “comprising”, the words “a” or “an” can mean one or more than one. The term “NRIP^{+/+} mice” used herein refers to a NRIP wild-type (WT) mice or generally means the mice whose spinal cord motor neurons are normal and do not have a loss of NRIP function. The term “NRIP^{-/-} mice” used herein refers to a NRIP knockout (KO) mice or means the mice whose spinal cord motor neurons are abnormal and have an abnormal NRIP function.

A loss of NRIP function generally means “reduced or abolished NRIP function.” A loss of function generally results from no expression or abnormal expression of NRIP-encoding gene, or inactivation of the gene product leading to less or no function of the gene.

As used herein, “NRIP” refers to a gene, protein or a nucleic acid encoding the protein. A “gene” refers to the smallest, independently functional unit of genetic material that can code for and drive the expression of a protein, e.g., NRIP, or whose presence or absence has a phenotypic consequence on a cell or organism. The term “an expression level of a NRIP”, as used herein, refers to the expression level of protein, RNA or DNA of NRIP. The NRIP gene, as used herein, is SEQ ID NO 1.

The present invention provides a method for evaluating a physiological state of motor neurons in a subject, comprising the following steps: (1) detecting an expression level of a nuclear receptor interaction protein (NRIP) in a biological sample from the subject; and (2) comparing the expression level of the NRIP to a control value for NRIP; wherein detecting a decrease of the expression level of the NRIP in the biological sample from the subject as compared to the control value for NRIP indicates an abnormal function of the motor neurons of the subject.

The expression level of the NRIP can be used as a biomarker for evaluating a normal or abnormal situation of the motor neurons. As used hereinafter, the term “biomarker” refers to a measured characteristic which can be used as an indicator of some biological state or condition. The term “biomarker” occasionally also refers to a substance whose presence indicates the existence of living organisms.

In one embodiment, the control value for NRIP is obtained from a biological sample of a normal subject. In a preferred embodiment, the control value for NRIP is a normal expression level of NRIP.

In one embodiment, the subject is an animal. Preferably, the subject is a mammal. Mammals include, but are not limited to, humans, primates, farm animals, sport animals, rodents and pets. More preferably, the subject is a human.

The term “motor neuron” applies to neurons located in the central nervous system (CNS) that project their axons outside the CNS to directly or indirectly control muscles. Motor neurons are efferent nerves which also are called effector neurons that carry signals from the spinal cord to the muscles to produce (effect) movement. Examples of motor neurons are primary motor neurons, alpha-motor neurons (α-motor neurons), beta-motor neurons (β-motor neurons) and gamma-motor neurons (γ-motor neurons). In one embodiment, the motor neurons are α-motor neurons.

In one embodiment, the abnormal function of the motor neurons is a decrease of a differentiation of the motor neurons. By “differentiation” is meant that the formation of cells expressing markers known to be associated with cells that are more specialized and closer to becoming terminally differentiated cells incapable of further division or differentiation. In a preferred embodiment, the abnormal function of the motor neurons is a degeneration of the motor neurons.

In addition, the abnormal function of the motor neurons is affected by the level of the acetylcholine (ACh) expression. In one embodiment, the abnormal function of the motor neurons is caused by a decrease of a choline acetyltransferase (ChAT) expression level. In a preferred embodiment, the decrease of the ChAT expression level is caused by the decrease of the expression level of the NRIP.

Based on above reason, the loss of the NRIP expression in the spinal cord motor neurons is sufficient to cause the abnormal function of the motor neurons, such as the degeneration or the decrease of the differentiation. Further, the abnormal function of the motor neurons causes a motor neuron disease. In one embodiment, the motor neuron disease is amyotrophic lateral sclerosis (ALS), primary lateral sclerosis (PLS), progressive muscular atrophy (PMA) or progressive bulbar palsy (PBP). In a preferred embodiment, the motor neuron disease is amyotrophic lateral sclerosis (ALS).

In another embodiment, the abnormal function of the motor neurons is a creation of amyotrophic lateral sclerosis (ALS). In a preferred embodiment, the abnormal function of the motor neurons is an increased risk of ALS.

As used herein, the term “amyotrophic lateral sclerosis (ALS)” refers to the ALS disease and ALS-like symptoms. The “ALS disease” means a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea). ALS is the most common, devastating, adult-onset neuromuscular degenerative disease. The term “ALS-like symptoms” shall generally mean “symptoms associated with ALS.” As used herein, “ALS-like symptoms comprises one or more than one of the following phenotypes: (a) kyphosis; (b) abnormal hind limb clasping; (c) deficiency in motor coordination and motor learning ability or deficiency in rotorad test; (d) motor neuron loss in the spinal cord; (e) astrocytosis in the spinal cord; (f) weight loss compared with a control rodent; and (g) accumulation of poly-ubiquitinated proteins in the spinal cord motor neurons.

The present invention can be used to evaluate or diagnose a risk of ALS in a subject. Accordingly, the present invention comprises measuring an expression level of a NRIP in a biological sample from the subject; and comparing the measured expression level of the NRIP with a control value for NRIP; wherein a decrease of the measured expression level of the NRIP from the biological sample as compared to the control value for NRIP is indicative of the risk of ALS in the subject.

In one embodiment, the biological sample is a blood sample. In a preferred embodiment, the biological sample is blood, serum or plasma.

In another embodiment, the expression level of the NRIP is detected by using immunoassay methodology. Preferably, the immunoassay methodology comprises enzyme-linked immunosorbent assay (ELISA), multiplex ligand binding, immunocytochemistry, fluorescence-activated cell sorting (FACS), or radioimmunoassay. More preferably, the ELISA comprises an indirect ELISA, a sandwich ELISA, or a competitive ELISA.

The present invention also provides a method for treating a motor neurons disease in a subject in need thereof, comprising administrating to said subject a pharmaceutically effective amount of composition comprising a nuclear receptor interaction protein (NRIP) and a pharmaceutically acceptable carrier.

In one embodiment, the subject is an animal. Preferably, the subject is a mammal. More preferably, the subject is a human. In one embodiment, the motor neurons are α-motor neurons, β-motor neurons or γ-motor neurons. In a preferred embodiment, the motor neurons are α-motor neurons.

The present invention can be used to treat, alleviate, ameliorate, relieve, delay onset of, inhibit progression of, reduce severity of, and/or reduce incidence of one or more symptoms or features of a disease, disorder, and/or condition induced by the motor neuron diseases.

In one embodiment, the motor neuron disease is amyotrophic lateral sclerosis (ALS), primary lateral sclerosis (PLS), progressive muscular atrophy (PMA) or progressive bulbar palsy (PBP). In a more preferred embodiment, the motor neuron disease is amyotrophic lateral sclerosis (ALS).

In one embodiment, the motor neuron disease is caused by an abnormal function of motor neurons. In a preferred embodiment, the abnormal function of motor neurons is a decrease of a differentiation of the motor neurons. In a more preferred embodiment, the abnormal function of the motor neurons is a degeneration of the motor neurons.

In one embodiment, the abnormal function of the motor neurons is affected by the expression level of the acetylcholine (ACh). In a preferred embodiment, the abnormal function of the motor neurons is caused by a decrease of a choline acetyltransferase (ChAT) expression level. In a more preferred embodiment, the decrease of the ChAT expression level is caused by a decrease of an expression level of the NRIP.

A “pharmaceutically effective amount” is an amount effective to prevent, lower, stop or reverse the development of, or to partially or totally alleviate the existing symptoms of a particular condition for which the subject being treated. Determination of a therapeutically effective amount is well within the capability of those skilled in the art.

The composition comprising the NRIP can be administered to the subject by many routes and in many regimens that will be well known to those in the art. In some embodiments, the NRIP is administered intravenously, intramuscularly, subcutaneously, topically, orally, or by inhalation. Through the digestive system and circulatory system, it is delivered to target locations.

The composition comprising the NRIP can be formulated for administration via sterile aqueous solution or dispersion, aqueous suspension, oil emulsion, water in oil emulsion, site-specific emulsion, long-residence emulsion, sticky-emulsion, microemulsion, nanoemulsion, liposomes, microparticles, microspheres, nanospheres, nanoparticles, minipumps, and with various natural or synthetic polymers that allow for sustained release. The compounds comprising the NRIP can also be formulated into aerosols, tablets, pills, sterile powders, suppositories, lotions, creams, ointments, pastes, gels, hydrogels, sustained-delivery devices, or other formulations used in drug delivery.

The pharmaceutically acceptable carriers are determined in part by the particular composition being administrated, as well as by particular method used to administer the composition. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a subject. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The present invention further provides a method of screening an agent for treating a motor neuron disease, comprising providing a test agent to a test sample from a subject suffering from the motor neuron disease, and measuring an expression level of a nuclear receptor interaction protein (NRIP) in test sample, wherein a decrease in the expression level of the NRIP in the test sample, relative to a control sample from a subject with no motor neuron disease, is indicative of the test agent being the agent for treating the abnormal function of the motor neurons.

In one embodiment, the subject and the subject with no motor neuron disease are animal. Preferably, the subject and the subject with no motor neuron disease are a mammal. More preferably, the subject and the subject with no motor neuron disease are a human.

In one embodiment, the test sample and the control sample are blood, serum, or plasma.

In one embodiment, the motor neuron disease is caused by the abnormal function of the motor neurons. In another embodiment, the motor neurons are α-motor neurons, β-motor neurons or γ-motor neurons. In a preferred embodiment, the motor neurons are α-motor neurons.

In one embodiment, the motor neuron disease is amyotrophic lateral sclerosis (ALS), primary lateral sclerosis (PLS), progressive muscular atrophy (PMA) or progressive bulbar palsy (PBP). In a more preferred embodiment, the motor neuron disease is amyotrophic lateral sclerosis (ALS).

In one embodiment, the motor neuron disease is caused by an abnormal function of motor neurons. In a preferred embodiment, the abnormal function of motor neurons is a decrease of a differentiation of the motor neurons. In a more preferred embodiment, the abnormal function of the motor neurons is a degeneration of the motor neurons.

In one embodiment, the abnormal function of the motor neurons is affected by the expression level of the acetylcholine (ACh). In a preferred embodiment, the abnormal function of the motor neurons is caused by a decrease of a choline acetyltransferase (ChAT) expression level. In a more preferred embodiment, the decrease of the ChAT expression level is caused by a decrease of an expression level of the NRIP.

In another embodiment, the expression level of the NRIP is measured by using immunoassay methodology. Preferably, the immunoassay methodology comprises enzyme-linked immunosorbent assay (ELISA), immunocytochemistry, fluorescence-activated cell sorting (FACS), multiplex ligand binding or radioimmunoassay. More preferably, the ELISA comprises an indirect ELISA, a sandwich ELISA, or a competitive ELISA.

### EXAMPLES

The examples below are non-limiting and are merely representative of various aspects and features of the present invention.

### Example 1

### The Loss of Motor Neurons in NRIP KO Mice is Age-Related but not Development Cause

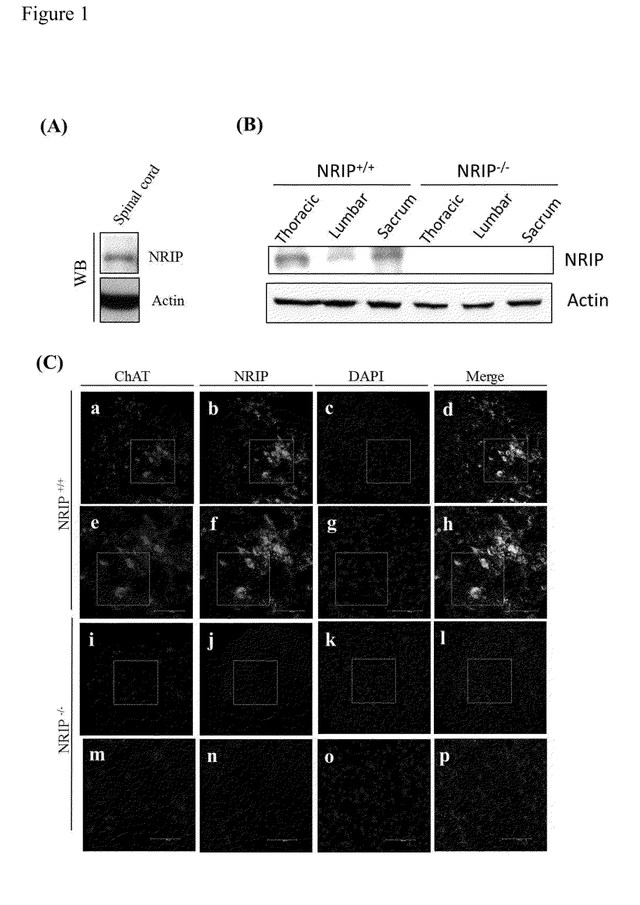

The present invention firstly examined the expression and the localization of nuclear receptor interaction protein (NRIP) in NRIP^{+/+} mice (i.e. NRIP wild-type mice or NRIP WT mice). By western blot analysis, NRIP was expressed in mice spinal cord (FIG. 1, parts A and B). Further, the present invention examined whether NRIP expressed in motor neurons (MNs). The immunofluorescence assay was performed for the expression of the NRIP and the choline acetyltransferase (ChAT, motor neuron biomarker) in lumbar spinal cord frozen tissue section. The results showed that NRIP (FIG. 1, part C, panels b, f) was co-localized with ChAT that expressed at MNs in ventral horn spinal cord (FIG. 1, part C, panels d, h). This finding suggested that the NRIP could play a role in MNs function.

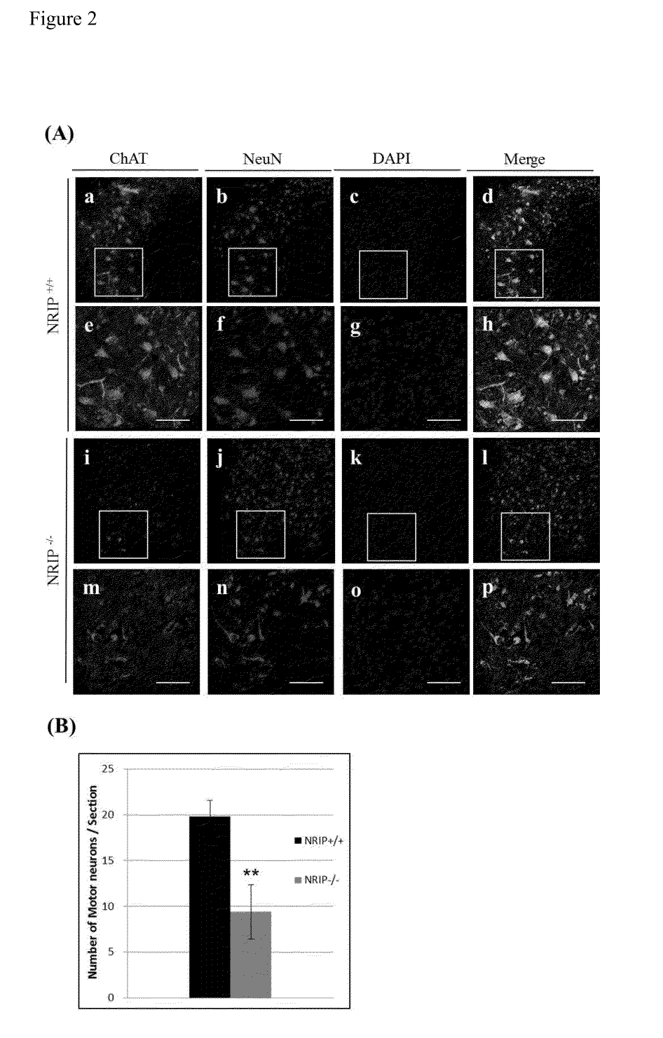

A single motor neuron innervates many muscle fibers. MNs were further subdivided to two groups: α-MNs (alpha-MNs) could innervate muscle fibers to produce muscle contraction and γ-MNs (gamma-MNs) maintained tautness of the muscle spindles. The size of alpha-MNs were much bigger (about 800 μm²) than gamma-MNs (about 200 μm²); and the α-MNs expressed ChAT and NeuN while γ-MNs expressed Err3. To distinguish the subtype of MNs, the double-labeling immunofluorescence assays were then performed to stain ChAT and neuronal nuclei (NeuN) in the spinal cord sections. The results showed that there was significant loss of α-MNs in NRIP^{-/-} mice (i.e. NRIP knockout mice or NRIP KO mice) (FIG. 2, part A, panels l, p, in yellow) compared to the NRIP^{+/+} mice (NRIP WT mice) (FIG. 2, part A, panel d, h, in yellow). Additionally, the remains of α-MNs in the ventral horn of NRIP KO mice showed broken and shrank phenotype compared to the NRIP WT mice. These results illustrated that the NRIP KO mice lost α-MNs, and the rest of MNs in adult NRIP KO mice were also unhealthy.

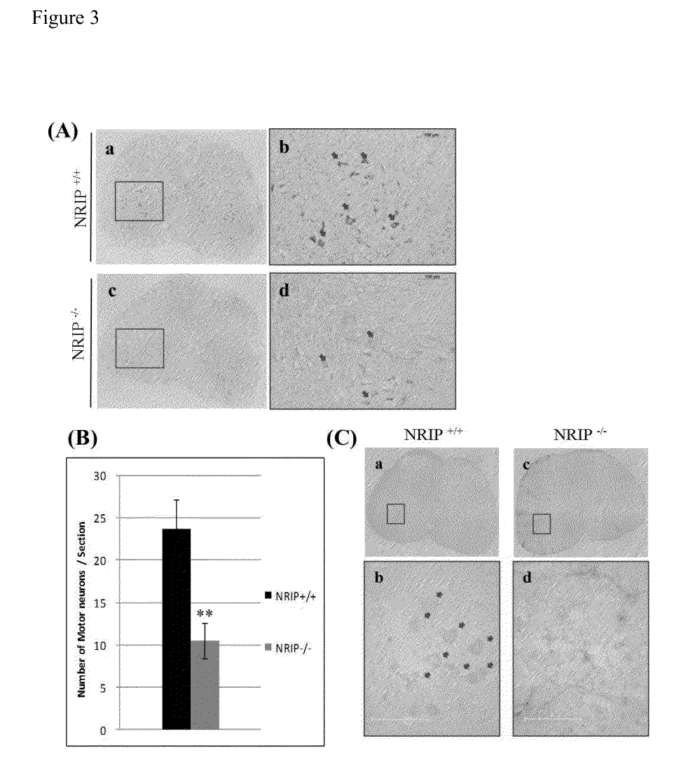

To examine whether MNs were lost in adult NRIP KO mice, the lumbar segments of spinal cords were dissected from 12 weeks to 16 weeks NRIP WT and KO mice (each group N=3). The whole lumbar segment of spinal cord was sectioned into slice in 30 μm thickness frozen section. Every fourth section was stained with 1% cresyl violet (15 sections total from each animal) and visualized by microscope. The sections were next counting manually by tracing the perimeter of each motor neuron in the ventral horn of gray matter. The red arrows indicated the presence of a nucleolus located within the nucleus surrounded by light blue-staining large cytoplasm that was counted as MN (Figure, part A). The statistical analysis from the part A of FIG. 3 showed that there was significant loss of MNs in NRIP KO mice compared to the NRIP WT mice (FIG. 3, part B). The immunofluorescence assay (IFA) results showed that the number of ChAT-positive MNs of NRIP WT mice was higher than NRIP KO mice, which consistent with the result in Niss1-staining (FIG. 3, part B). Additionally, the distribution of MNs in lumbar spinal cord was further confirmed by the ChAT staining immunohistochemistry assay as shown in the part C of Figure.

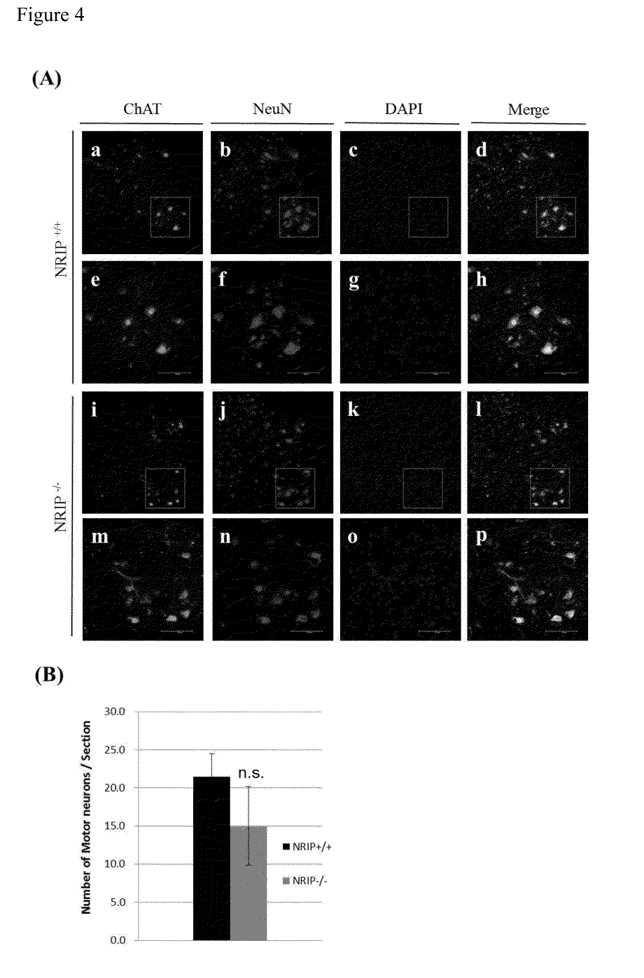

To investigate whether the deficient of MN in NRIP KO mice was occurred in the postnatal stage before adult, the spinal cords of the six weeks old postnatal NRIP WT and KO mice were isolated and analyzed by immunofluorescence assay for the expression of the ChAT and the NeuN. The results showed that the number and size of MNs in NRIP KO mice (FIG. 4, part A, panels l, p) were the same as NRIP WT mice (FIG. 4, part A, panels d, h). There was no significance by the statistical analysis between NRIP WT mice and NRIP KO mice at age 6 weeks (FIG. 4, part B). It indicated that the deficiency of MNs in NRIP KO mice was age-related but not development cause.

### The Gliosis in NRIP KO Mice

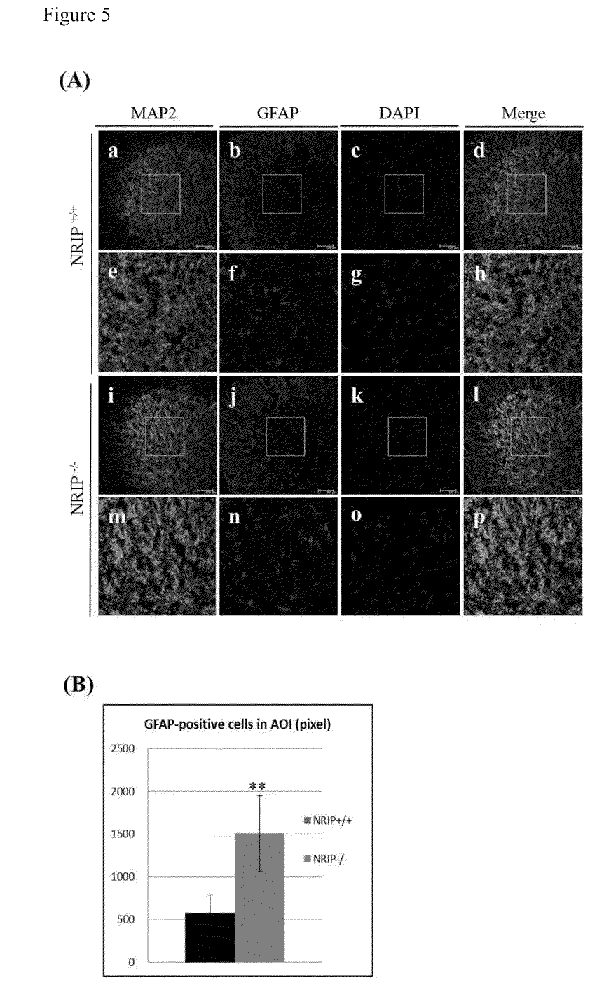

Gliosis, known as glia scar formation, is an inflammatory response followed by neuron injury in the central nervous system (CNS). After neuron injury, astrocytes and microglia can be activated/proliferated and then promptly move to the damaged lesions, which undergo morphological changes and increase synthesis of intermediate protein such as glial fibrillary acidic protein (GFAP) to separate them from the adjacent normal nervous tissue by forming a physical and molecular barrier. The current reports mentioned that the mouse model of ALS was caused by the loss of MNs in spinal cord in which gliosis occurred due to astrocytes produced GFAP forming the intermediate filament wherein the up-regulation of GFAP was regarded as the hallmark of astrogliosis. To examine whether astrogliosis occurred in NRIP^{-/-} mice (NRIP KO mice), the GFAP and microtubule-associated protein 2 (MAP2; neuron dendrite marker) were stained by immunofluorescence assay to examine the astrogliosis and the growth of dendrites respectively. The results showed that the GFAP of NRIP KO mice (FIG. 5, part A, panels j, n) was expressed significantly higher than NRIP^{+/+} mice (NRIP WT mice) (FIG. 5, part A, panels b, f). The calculated results showed that GFAP positive signals in NRIP KO mice (FIG. 5, part B) were twice more than that in NRIP WT mice while there was no significant difference in MAP2 between NRIP WT and KO mice (FIG. 5, part A, panels a, e, i, m). In sum, the astrogliosis occurred in adult NRIP KO mice.

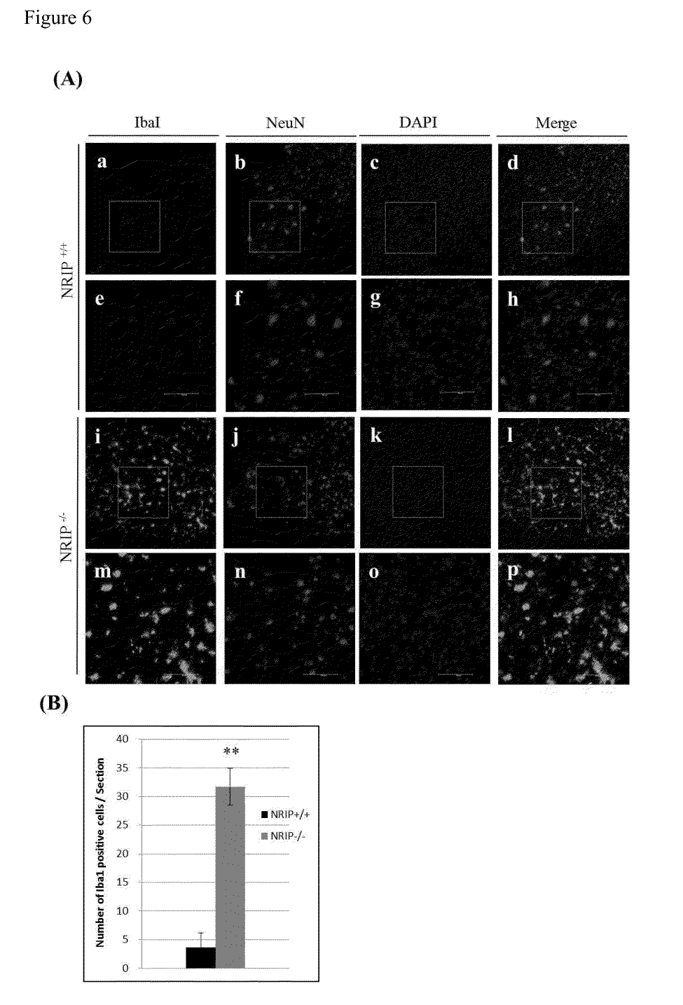

Microglia is the resident macrophage in the CNS and serves as the first line of defense in the CNS; any insult to the CNS homeostasis will induce a rapid change in microglia morphology, gene expression and functional behavior. These phenomenons are called microgliosis. After neuron injury, the activated microglia will migrate to the site of lesion and form a dense border to seal the lesion and block the spread of damage within minutes. The present invention then examined the effect of microgliosis by immunofluorescence assay when the NRIP was null. Ionized calcium-binding adapter molecule 1 (Iba1) which expressed specifically in microglia served as the biomarker for tracing the activation of microglia. The results showed that Iba1 was strongly expressed in NRIP KO mice (FIG. 6 part A, panels i, m) but not in NRIP WT mice (FIG. 6, part A, panels a, e); and the quantitative analysis of Iba1 positive microglia cells in ventral horn spinal cord from the part A of FIG. 6 showed significant difference by student's t test between NRIP WT and NRIP KO mice (FIG. 6, part B). It indicated that gliosis occurred in adult NRIP KO mice. Hence, the NRIP played a role for protection of spinal cord neurons.

### The Mechanism of NRIP Role in ChAT in Cholinergic Cell Line NG108.

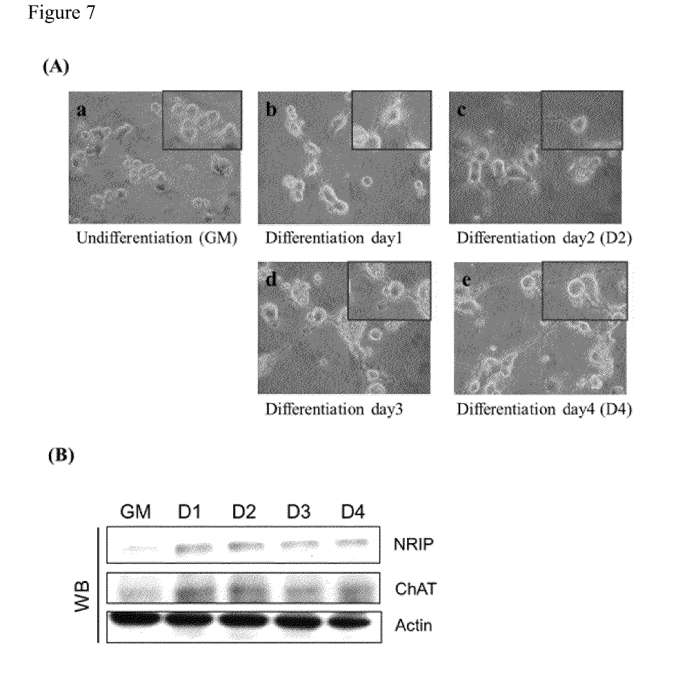

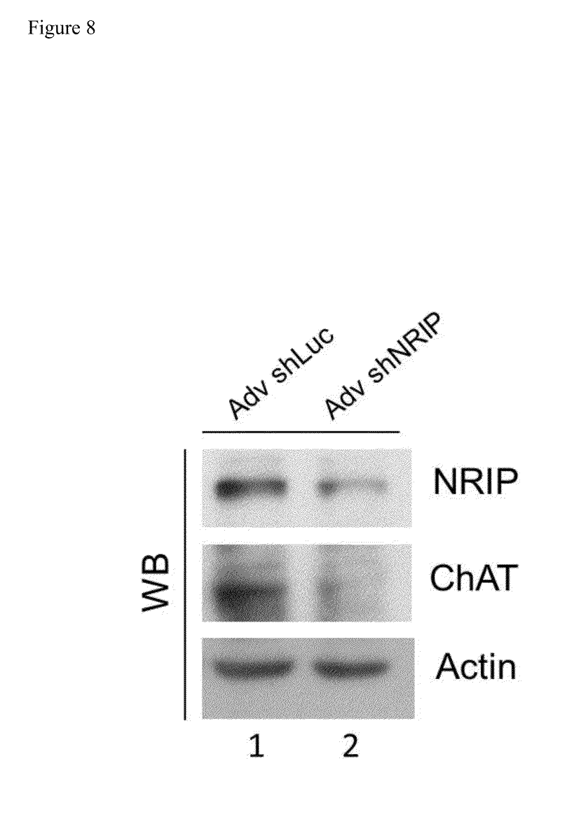

The present invention further investigated how NRIP regulated MNs in cholinergic neuron cell line (NG108). NG108 is a hybrid cell line of mouse neuroblastoma (N18TG-2) and rat glioma (C6BU-1) that is widely used in in vitro experiments instead of primary-cultured neurons. NG108 cells generally proliferate well in the culture medium, but stop proliferating and frequently show the growth of neurites in the presence of dibutyryl cyclic-AMP (dbcAMP). The expression of the ChAT in differentiated NG108 served as a model for mimic MNs. The present invention then seeded NG108 cells in low density for motor neuron differentiation. After induced by 1 mM dbcAMP followed with low serum medium, cells were differentiated and the neurites appeared. The results showed that short neuritis appeared one day after induced differentiation (FIG. 7, part A). Dendrites kept growing and reached to nearby cells at about three days after differentiation. Neurites were all cross linking to nearby cells at day four after differentiation. The results showed that both the ChAT and the NRIP were both up-regulated during the neuron differentiation (FIG. 7, part B). Furthermore, the ChAT was reduced upon deprivation of NRIP expression (FIG. 8). Therefore, it indicated that NRIP involved in motor neuron differentiation; and NRIP knockout mice could serve as a mouse model for ALS.

One skilled in the art readily appreciates that the present invention is well adapted to carry out the objects and obtain the ends and advantages mentioned, as well as those inherent therein. The animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for evaluating a physiological state of motor neurons in a subject, comprising the following steps:
(1) detecting an expression level of a nuclear receptor interaction protein (NRIP) in a biological sample from the subject; and
(2) comparing the expression level of the NRIP to a control value for NRIP; wherein detecting a decrease of the expression level of the NRIP in the biological sample from the subject as compared to the control value for NRIP indicates an abnormal function of the motor neurons of the subject.

2. The method of claim 1, wherein the abnormal function of the motor neurons is a decrease of a differentiation of the motor neurons.

3. The method of claim 1, wherein the abnormal function of the motor neurons is a degeneration of the motor neurons.

4. The method of claim 1, wherein the abnormal function of the motor neurons is caused by a decrease of a choline acetyltransferase (ChAT) expression level.

5. The method of claim 4, wherein the decrease of the ChAT expression level is caused by the decrease of the expression level of the NRIP.

6. The method of claim 1, wherein the abnormal function of the motor neurons further causes a motor neuron disease.

7. The method of claim 6, wherein the motor neuron disease is amyotrophic lateral sclerosis (ALS).

8. The method of claim 7, wherein the abnormal function of the motor neurons is a creation of ALS.

9. The method of claim 7, wherein the abnormal function of the motor neurons is an increased risk of ALS.

10. The method of claim 1, wherein the subject is a mammal.

11. The method of claim 1, wherein the subject is a human.

12. The method of claim 1, wherein the motor neurons are α-motor neurons.

13. The method of claim 1, wherein the control value for NRIP is obtained from a biological sample of a normal subject.

14. The method of claim 1, wherein the biological sample is blood, serum or plasma.

15. The method of claim 1, wherein the expression level of the NRIP is detected by using immunoassay methodology.

16. The method of claim 15, wherein the immunoassay methodology comprises immunocytochemistry, enzyme-linked immunosorbent assay (ELISA), fluorescence-activated cell sorting (FACS), multiplex ligand binding or radioimmunoassay.
